(19) United States
(12) Patent Application Publication
(10) Pub. No.: US 2024/0279637 A1
(43) Pub. Date: Aug. 22, 2024

(54) EXTRACTION OF PLASMID DNA

(71) Applicant: Cytiva Sweden AB, Uppsala (SE)

(72) Inventors: Hans Blom, Uppsala (SE); Markus Pitkanen, Uppsala (SE); Linda Hagman, Uppsala (SE); Annika Morrison, Uppsala (SE)

(21) Appl. No.: 18/693,116

(22) PCT Filed: Sep. 30, 2022
(86) PCT No.: PCT/EP2022/077388

§ 371 Date: Mar. 18, 2024

(30) Foreign Application Priority Data
Oct. 8, 2021 (GB) 2114428.2

(51) Int. Cl. C12N 15/10; C12M 1/00; C12M 1/34

(52) U.S. Cl. CPC C12N 15/1003; C12M 23/14; C12M 41/12; C12M 41/26; C12M 47/06

(57) ABSTRACT

A method, a system and a use of a single use mixing bag for performing alkaline lysis, neutralisation and clarification of bacterial cells for extraction of plasmid DNA. The method comprises providing a suspension of bacterial cells and providing alkaline lysis solution into a single use mixing bag for performing an alkaline lysis of said bacterial cells, wherein said single use mixing bag comprises an integrated mixer. The method comprises furthermore mixing of the content in the single use mixing bag by the integrated mixer during the alkaline lysis and stopping the alkaline lysis by the addition of a neutralisation solution into the single use mixing bag and clarifying the content in the single use mixing bag after the alkaline lysis has been performed by the addition of a flocculate lifting agent into the single use mixing bag, whereby at least two phases are separated inside the single use mixing bag, whereby one of said phases is a neutralised and clarified alkaline solution comprising plasmid DNA. The neutralised and clarified alkaline solution is removed from the single use mixing bag and filtered.

### TECHNICAL FIELD OF THE INVENTION

The present invention relation to a method and a system for performing alkaline lysis, neutralisation and clarification of bacterial cells for extraction of plasmid DNA.

### BACKGROUND

A known method for extracting plasmid DNA from bacteria cells, such as E-coli, comprises a first step of lysing the bacterial cells in an alkaline lysis process. An alkaline lysis solution is provided to a suspension of bacterial cells whereby a lysation of the cells is started. The lysation is stopped after a suitable time by the addition of a neutralisation solution which results in the immediate formation of a precipitation/flocculate. The flocculate has a density similar to the surrounding solution and can be seen distributed in the whole volume. The most common methods for flocculate removal involves filtration and/or centrifugation techniques. The wide size distribution of the particulate material in the flocculate generally does not allow a single step of filtration due to the high risk of filter clogging. Rather, to accomplish a sufficient clarification, combinations of different filtration steps, filter aids, such as Diatomaceous earth, with or without a preceding centrifugation step, is commonly used.

In IP.com Number: IPCOM000146505D a process for simplifying flocculate removal is disclosed. Lysis is performed according to the typical procedure described above but Ammonium hydrogen carbonate is added after neutralization which causes the flocculate to float to the surface and form a stable layer. The addition of Ammonium hydrogen carbonate produces carbon dioxide gas bubbles that rise to the surface carrying with it the flocculate material which forms a floating layer at the liquid surface. The resulting solution is clear and mainly devoid of particulate material and can be decanted.

There is still a need to improve the method for extraction of plasmid DNA.

### SUMMARY

An object of the invention is to provide an improved method and system for extraction of plasmid DNA.

A further object of the invention is to provide a method and system for extraction of plasmid DNA which is suitable for performing an automated process in a closed system.

This is achieved by a method, a use and a system as defined in the independent claims.

According to one aspect of the invention a method for performing alkaline lysis, neutralisation and clarification of bacterial cells for extraction of plasmid DNA is provided. Said method comprises the steps of:
providing a suspension of bacterial cells and providing alkaline lysis solution into a single use mixing bag for performing an alkaline lysis of said bacterial cells, wherein said single use mixing bag comprises an integrated mixer;
mixing the content in the single use mixing bag by the integrated mixer during the alkaline lysis;
stopping the alkaline lysis by adding a neutralisation solution into the single use mixing bag;
clarifying the content in the single use mixing bag after the alkaline lysis has been performed by adding a flocculate lifting agent into the single use mixing bag, whereby at least two phases are separated inside the single use mixing bag, whereby one of said phases is a neutralised and clarified alkaline solution comprising plasmid DNA;
removing the neutralised and clarified alkaline solution from the single use mixing bag;
filtering the neutralised and clarified alkaline solution.

According to another aspect of the invention a use of a single use mixing bag for performing alkaline lysis, neutralisation and clarification of bacterial cells according to the method of the invention is provided, wherein said single use mixing bag comprises at least one inlet, at least one outlet, a vent valve device and an integrated mixer.

According to another aspect of the invention a system for performing alkaline lysis, neutralisation and clarification of bacterial cells for extraction of plasmid DNA is provided wherein said system comprises:
a single use mixing bag which comprises at least one inlet, at least one outlet, of which one is a clarified phase outlet, a vent valve device and an integrated mixer;
at least one inlet fluid line connected to at least one of the at least one inlet of the single use mixing bag and having at least one connector for connection to fluid sources comprising fluids to be added to the single use mixing bag;
at least one inlet pump connected to the at least one inlet fluid line;
an outlet fluid line connected to the clarified phase outlet of the single use mixing bag;
at least one outlet pump connected to the outlet fluid line;
a collection bag connected to the outlet fluid line for collection of a neutralised and clarified alkaline solution removed from the single use mixing bag via the clarified phase outlet;
a filter connected to the outlet fluid line and provided between the clarified phase outlet and the collection bag such that a content removed from the clarified phase outlet of the single use mixing bag is filtered through the filter on its way to the collection bag; and
a control system connected to the at least one inlet pump, the at least one outlet pump and to a mixer controller which can control the integrated mixer of the single use mixing bag, wherein said control system is configured for controlling the at least one inlet pump, the at least one outlet pump and the integrated mixer in the single use mixing bag to pump content into the single use mixing bag, pump content out from the single use mixing bag and mix the content in the single use mixing bag according to the method of the invention.

Hereby, by using a single use mixing bag for the lysis, neutralisation and clarification steps in the method for extraction of plasmid DNA the method can be performed in a closed system and the method can easily be automated. The mixing can be performed by an integrated mixer provided in the single use mixing bag. This mixer can for example be controlled by magnetic force from outside the single use mixing bag and hereby the content in the bag can be mixed without any direct mechanical contact from outside the bag. Hereby a closed handling can be kept during the extraction of plasmid DNA, which is advantageous. When handling bacterial cells it is important to avoid contamination of the surrounding environment and the use of a single use mixing bag in this lysis process will allow a closed process which will decrease risks for contamination considerably. Furthermore, the process can easily be automated with the method and system according to the invention. Pumps can be controlled to pump content into and out from the single use mixing bag and the mixer can be controlled to mix the content inside the bag. Hereby the use of a single use mixing bag is advantageous for performing extraction of plasmid DNA according to this method. The use of a single use mixing bag provides for a quicker process with better precision than previous known methods which are involving more manual interaction. Furthermore, a more controlled mixing can be provided thanks to the integrated mixer compared to a manual mixing.

In some embodiments of the invention the step of clarifying comprises adding a solution of a flocculate lifting agent comprising a salt which releases gas in acidic environment into the single use mixing bag by pumping the solution of flocculate lifting agent by a pump into an inlet of the single use mixing bag. Hereby the gas bubbles will rise to an upper surface of the content in the mixing bag and carry flocculate towards the upper surface whereby an upper phase comprising flocculate and a lower phase comprising a neutralised and clarified alkaline solution will be formed in the mixing bag.

In some embodiments of the invention the method further comprises the step of sensing one or more parameters of the content in the single use mixing bag and/or of the content in an inlet fluid line and/or outlet fluid line connected to the single use mixing bag by one or more sensors positioned in one or more positions inside the single use mixing bag and/or in connection with the inlet fluid line and/or the outlet fluid line. In some embodiments of the invention said single use mixing bag comprises at least one sensor which is configured for sensing one or more parameters of the content in the single use mixing bag, such as one or more of pH, temperature, turbidity and conductivity. In some embodiments of the invention the method further comprises controlling pumping of contents into and out from the single use mixing bag and/or mixing velocity of the integrated mixer in the single use mixing bag based on the sensed parameters detected by the one or more sensors.

Hereby sensed parameters can be used for control, adjustment and optimisation of the method and system by controlling pumps and/or the integrated mixer. The sensed parameters can also be used for statistics and reporting.

In some embodiments of the invention said single use mixing bag is a flexible, plastic bag which is sterilized and provided with aseptic connectors to the at least one inlet and the at least one outlet. Hereby the single use mixing bag can easily be used for performing alkaline lysis, neutralisation and clarification of bacterial cells for extraction of plasmid DNA according to the method of the invention. The single use mixing bag can easily be connected aseptically to fluid sources.

Further embodiments are described in the detailed description and in the dependent claims.

### BRIEF DESCRIPTION OF THE DRAWINGS

FIG. 1 is a schematic drawing of a system for performing alkaline lysis, neutralisation and clarification of bacterial cells for extraction of plasmid DNA according to one embodiment of the invention.

FIG. 2 is a flow chart of a method for performing alkaline lysis, neutralisation and clarification of bacterial cells for extraction of plasmid DNA according to one embodiment of the invention.

### DETAILED DESCRIPTION OF EMBODIMENTS

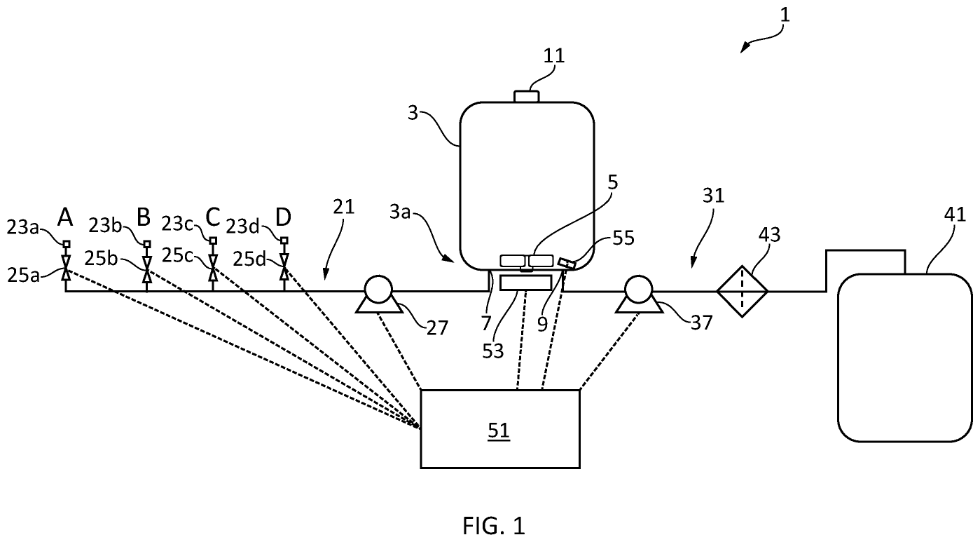

FIG. 1 is a schematic drawing of a system 1 for performing alkaline lysis, neutralisation and clarification of bacterial cells for extraction of plasmid DNA according to one embodiment of the invention. According to the invention a single use mixing bag 3 is used for the extraction of plasmid DNA. The single use mixing bag 3 can be made from a flexible and plastic material and is supposed to be disposed after the first use. The single use mixing bag 3 comprises an integrated mixer 5 which can be controlled from outside the bag without any direct mechanical contact, i.e. without any contact with the external environmental and without risk for contamination. The integrated mixer 5 can for example be a magnetic impeller 5 which is controlled by magnetic force from outside the mixing bag without any direct mechanical contact. Hereby the single use mixing bag 3 is suitable for use in a closed system which can handle content without risking contamination of the external environment outside the system. The single use mixing bag 3 comprises furthermore at least one inlet 7 and at least one outlet 9 which in some circumstances could be the same inlet/outlet (i.e. only one inlet/outlet could in some examples be provided to the single use mixing bag). However, in the example shown in FIG. 1 one separate inlet 7 and one separate outlet 9 are provided. The number of inlets and outlets provided in the mixing bag 3 could however also be more than one. The outlet 9 is here called a clarified phase outlet 9 as it is intended for transferring a neutralised and clarified alkaline solution out from the mixing bag 3 through this clarified phase outlet 9 after a clarification step according to the method of the invention has been performed. Both the inlet 7 and the clarified phase outlet 9 are in this embodiment provided in a bottom part 3a of the single use mixing bag 3. The integrated mixer 5 is also provided in the bottom part 3a of the single use mixing bag 3. Hereby, thanks to the positioning of the inlet 7 close to the integrated mixer 5 it is assured that mixing of content which is introduced through the inlet 7 will be performed effectively by the integrated mixer 5. The position of the clarified phase outlet 9 in a bottom part 3a of the mixing bag 3 will be discussed further down in the description, however this position will allow an effective removal of only the lower phase of the content in the SU bag, which lower phase will be a neutralised and clarified alkaline solution comprising plasmid DNA after the lysis, neutralisation and clarification. The single use mixing bag 3 comprises furthermore suitably a vent valve device 11. The vent valve device 11 is provided for allowing release of excessive gas during for example the clarification step and/or for allowing release of potential foam generated during the flock lifting as will be described in more detail below. The single use mixing bag 3 may be sterilized and provided with aseptic connectors to its inlets 7 and outlets 9 at delivery. A user may connect the single use mixing bag 3 with the other parts of the system and to a control system as will be described below. The system according to the invention comprises furthermore at least one inlet fluid line 21 which is connected to or is configured to be connected to (for example by comprising one or more aseptic connectors) at least one of the at least one inlet 7 of the single use mixing bag 3. The inlet fluid line 21 comprises also at least one fluid source connector 23a-d for connection to at least one fluid source A, B, C, D comprising fluids to be added to the single use mixing bag 3. The fluid source connectors 23a-d could in some embodiments of the invention be aseptic connectors. In the embodiment shown in FIG. 1 four fluid source connectors 23a-d are provided and four different fluid sources A, B, C, D can be connected to the inlet fluid line 21, one to each of the fluid source connectors 23a-d. In another embodiment separate inlet fluid lines 21 could be used for each fluid source A, B, C, D and separate inlets 7 of the single use mixing bag could also be used for each fluid source A, B, C, D. I.e. more than one inlet 7 could be provided to the single use mixing bag 3 and the different fluids to be added to the single use mixing bag 3 can in some embodiments be added via different inlets 7. In further another embodiment two or more of the fluid sources A, B, C, D could be connected to the same fluid source connector 23a-d after each other in the process. In such an embodiment the number of fluid source connectors 23a-b of the inlet fluid line 21 can be less than four, such as one, two or three.

At least one inlet pump 27 is also provided in the system 1. The inlet pump 27 is connected to the at least one inlet fluid line 21 and is configured for pumping fluid from the fluid sources A, B, C, D into the single use mixing bag 3 via the at least one inlet 7.

The system according to the invention comprises furthermore at least one outlet fluid line 31 which is connected to or is configured to be connected to (for example by comprising one or more aseptic connectors) the clarified phase outlet 9 of the single use mixing bag 3. At least one outlet pump 37 is also provided in the system 1. Said outlet pump 37 is connected to the outlet fluid line 31. The outlet pump 37 is configured for pumping a content out from the single use mixing bag 3 via the clarified phase outlet 9. The system 1 comprises furthermore a collection bag 41 connected to the outlet fluid line 31 for collection of a neutralized and clarified alkaline solution removed from the single use mixing bag 3 via the clarified phase outlet 9. The system 1 comprises furthermore a filter 43 connected to the outlet fluid line 31 and provided between the clarified phase outlet 9 and the collection bag 41 such that a content removed from the clarified phase outlet 9 of the single use mixing bag 3 is filtered through the filter 43 on its way to the collection bag 41. The filter 43 can for example be a depth filter or a dead end filter. In some embodiments the filter is a depth filter which effectively will remove remaining flocculate and/or particulate material.

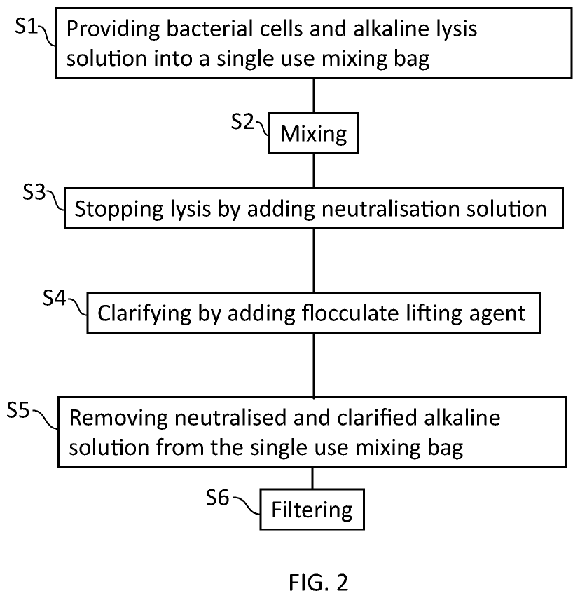

The system 1 comprises furthermore a control system 51 which is connected to the at least one inlet pump 27, the at least one outlet pump 37 and to a mixer controller 53 which can control the integrated mixer 5 of the single use mixing bag 3 without direct contact, wherein said control system 51 is configured for controlling the at least one inlet pump 27, the at least one outlet pump 37 and the integrated mixer 5 in the single use mixing bag 3 to pump content into the single use mixing bag 3, pump content out from the single use mixing bag 3 and mix the content in the single use mixing bag 3 according to the method of the invention which will be described below with reference to FIG. 2. The control system 51 can possibly also be connected to any valves 25a-d which may be provided in the system. Valves 25a-d are shown provided in connection with the connectors 23a-d to which the fluid sources A, B, C, D may be connected. Hereby it can be controlled which fluid source A, B, C, D is connected to the inlet fluid line 21.

FIG. 2 is a flow chart of a method according to one embodiment of the invention. The method is a method for performing alkaline lysis, neutralisation and clarification of bacterial cells for extraction of plasmid DNA. The method steps are described in order below:

S1: Providing a suspension of bacterial cells and providing alkaline lysis solution into a single use mixing bag 3 for performing an alkaline lysis of said bacterial cells, wherein said single use mixing bag 3 comprises an integrated mixer 5. As an example the suspension of bacterial cells can be represented by A in FIG. 1 and the alkaline lysis solution can be represented by B in FIG. 1. In some embodiments of the invention the suspension of bacterial cells is first added to the single use mixing bag 3 and then the alkaline lysis solution is added to the single use mixing bag 3. As shown in FIG. 1 the suspension of bacterial cells, A, and the alkaline lysis solution, B, are added via the inlet fluid line 21 into the mixing bag 3 though the inlet 7. The inlet pump 27 is used for pumping the suspension of bacterial cells, A, and the alkaline lysis solution, B, through the inlet fluid line 21. A first volume, V1, of suspension of bacterial cells is in some embodiments first pumped into the single use mixing bag 3 and then a second volume, V2, of an alkaline lysis solution is pumped into the single use mixing bag 3. The first and second volumes, V1 and V2 are in some embodiments equal but they may also be different depending on the concentrations of the solutions. The step of pumping the second volume, V2, of an alkaline lysis solution into the mixing bag 3 is suitably performed during a limited time period. The dimensions and performance of the inlet pump 27 and the inlet fluid line 21 can be chosen for allowing a quick transfer with a defined time range. For example, the time period needed for pumping all the alkaline lysis solution into the mixing bag 3 can be controlled to be between 0.5-3 minutes or between 0.5-1.5 minutes. In some embodiments the method comprises the step of mixing the content inside the mixing bag during the transfer of alkaline lysis solution into the mixing bag. This step of mixing may be performed during at least a part of said time period during which alkaline lysis solution is pumped into the single use mixing bag 3.

The suspension of bacterial cells could for example be a suspension of E. coli cells. The alkaline lysis solution can for example be 0.1-0.5 M NaOH+0.1-1.0% SDS.

S2: Mixing the content in the single use mixing bag 3 by the integrated mixer 5 during the alkaline lysis. Alkaline lysis of the bacterial cells will be initiated as soon as the alkaline lysis solution pumped into the mixing bag 3 comes into contact with the suspension of bacterial cells that has already been provided into the mixing bag 3. When all the alkaline lysis solution has been pumped into the mixing bag 3 the lysis may need to continue for a defined time range (minutes) and mixing will be performed during this time or at least during a part of this time.

S3: Stopping the alkaline lysis by adding a neutralisation solution into the single use mixing bag 3. The neutralisation solution can be illustrated by C in FIG. 1. The step of stopping the alkaline lysis by adding a neutralisation solution into the single use mixing bag 3 can be performed by adding a neutralisation solution consisting of 2.5-10 M potassium acetate having a pH of 4.5-6.0 and a temperature between 1-8° C. This step may also comprise the step of mixing the content in the single use mixing bag 3 by the integrated mixer 5 during the time it takes for the addition of the neutralisation solution. However, it may be suitable to use a lower mixing velocity during this step of stopping the alkaline lysis compared to a mixing velocity during the alkaline lysis. This is in order to avoid fragmentation of genomic DNA. The time for transferring the neutralisation solution, C, into the single use mixing bag 3 by the inlet pump 27 can suitably be kept as short as possible. A total time for the alkaline lysis as counted from start of addition of alkaline lysis solution until end of addition of neutralisation solution can suitably be kept less than 10 minutes or less than 6 minutes. Hereby there is lower risk for fragmentation of genomic DNA which fragments may be hard to separate from the plasmid DNA in further downstream process steps involving for example chromatography of membrane adsorbers. The mixing velocity may also need to be adjusted appropriately during both the alkaline lysis step and the neutralisation step to avoid fragmentation/disintegration of genomic DNA due to the shear forces that may occur during mixing.

The addition of a neutralisation solution for stopping the alkaline lysis will give rise to a formation of a precipitate/flocculate in the content in the mixing bag 3. This flocculate has a density which is similar to the surrounding solution and may be distributed in the whole volume.

S4: Clarifying the content in the single use mixing bag 3 after the alkaline lysis has been performed by adding a flocculate lifting agent into the single use mixing bag 3. The flocculate lifting agent can be a solution and can be illustrated by D in FIG. 1. A flocculate lifting agent is in this invention defined as an agent, suitably a solution of salt, which will produce gas bubbles in acidic environment. Said gas bubbles will rise to an upper surface of the content in the mixing bag and carry the flocculate which has been produced during the step of stopping the alkaline lysis towards the surface. The flocculate is hereby lifted with the gas bubbles and will form a layer or a cake as an upper phase of the content in the mixing bag 3. Hereby at least two phases are separated inside the single use mixing bag 3 during this step of clarifying and one of said phases is a neutralised and clarified alkaline solution comprising plasmid DNA while another of said phases is the flocculate forming a semi-solid upper layer containing particulate material. The flocculate lifting agent can be a salt which produce gas in acidic environment such as for example ammonium hydrogen carbonate (AHC), potassium hydrogen carbonate, sodium hydrogen carbonate or similar agents. Ammonium hydrogen carbonate will be very effective for the separation into at least two phases. It will effectively bring the flocculate to an upper phase of the content in the mixing bag forming a layer of flocculate material at the upper surface. Small amounts of flocculate may still be left in the neutralised and clarified alkaline solution which is provided below the upper phase of flocculate in the mixing bag 3.

The step of clarifying may comprise to add the flocculate lifting agent as a solution of for example ammonium hydrogen carbonate or a similar flocculate lifting agent into the single use mixing bag 3 by pumping the solution of flocculate lifting agent by a pump which may be the same inlet pump 27 as used for the addition of bacterial cells, alkaline lysis solution and neutralisation solution or another inlet pump. The flocculate lifting agent is added into the mixing bag 3 via an inlet 7 of the single use mixing bag 3 which may be the same inlet 7 as used for adding the other fluids or it may be another inlet. The solution of a flocculate lifting agent may for example comprise 60-150 g salt, such as AHC, per litre. A volume and a concentration of the solution of the flocculate lifting agent which for example can be a solution of a salt such as ammonium hydrogen carbonate, potassium hydrogen carbonate or sodium hydrogen carbonate, can suitably be provided such that a concentration of the salt in the combined content in the single use mixing bag after the addition of the salt solution is within an interval of 2-15 g salt/litre.

S5: Removing the neutralised and clarified alkaline solution from the single use mixing bag 3. The neutralised and clarified alkaline solution may be separated in the clarification step into a lower phase which can be removed from the mixing bag 3 through the clarified phase outlet 9 by the outlet pump 37 and through the outlet fluid line 31.

S6: Filtering the neutralised and clarified alkaline solution. On the way from the mixing bag 3 the neutralised and clarified alkaline solution will pass a filter 43. This can be an appropriate filter such as for example a depth filter or a dead end filter, which effectively will remove remaining particulate material in the neutralised and clarified alkaline solution. The filtered and neutralised and clarified alkaline solution may then be collected in a collection bag 41.

The method according to the invention may further comprise the step of sensing one or more parameter of the content in the mixing bag 3 or content in the inlet or outlet fluid lines 21, 31. The parameters could be one or more of for example pH, temperature, turbidity and conductivity. The parameters can be sensed by one or more sensors 55 positioned in one or more positions inside the single use mixing bag 3 or in direct association with the inlet or outlet fluid lines 21, 31. Hereby the single use mixing bag 3 may in some embodiments of the invention comprise at least one sensor 55 which is configured for sensing one or more parameters of the content inside the mixing bag 3, such as for example one or more of pH, temperature, turbidity and conductivity. If for example a turbidity sensor is provided in vicinity of the clarified phase outlet 9 an outtake through the clarified phase outlet 9 can be monitored such that only the clarified phase is removed through the clarified phase outlet 9. The outlet pump 37 may be controlled in dependence of a sensor output and may stop pumping when an output from a sensor 55 positioned in vicinity of the clarified phase outlet 9 detects the presence of particulate material above a predefined threshold which may indicate that the content is not the neutralised and clarified alkaline solution but rather the flocculate.

The control system 51 may be configured for controlling the at least one outlet pump 37 to pump content out from the single use mixing bag 3 based on a sensed property from a sensor 55 positioned in vicinity of the clarified phase outlet 9. Hereby it can be assured that only the neutralised and clarified alkaline solution is removed from the single use mixing bag 3 and forwarded to the collection bag 41 via the filter 43. Hereby it can effectively be assured that no or a minimum of particulate material is transferred out from the single use mixing bag 3 and through the filter 43. Such particulate material could otherwise cause clogging of the filter 43 which could cause problems for an automated process. By using sensors 55 the process can be automated effectively. For example temperature and/or pH and/or turbidity and/or conductivity sensors can be provided in the mixing bag 3 either for documentation and statistics or also for controlling and feedback control of the system. The control system 51 may use sensor output for control of mixer speed and/or for control of pumping fluids into the mixing bag 3 from the connected fluid sources.

The method may further comprise the step of releasing excessive gases and potential foam produced during for example the step of clarifying via a vent valve device 11 provided in the single use mixing bag 3.

The method according to the invention may be an automated method controlled by a control system 51 to which said single use mixing bag 3 and pumps 27, 37 are connected. Hereby a closed and automated system is provided by which plasmid DNA can effectively be extracted. A closed single use system for extraction of plasmid DNA is suitable for use in processes that may require GMP classified environments or similar environments in order to minimize the risk of contamination of the external environment from the bacteria used.

The invention furthermore relates to use of a single use mixing bag 3 for performing alkaline lysis, neutralisation and clarification of bacterial cells as described above with reference to FIGS. 1 and 2. Such a single use mixing bag 3 comprises as described above at least one inlet 7, at least one outlet 9, a vent valve device 11 and an integrated mixer 5. The single use mixing bag 3 can be a flexible, plastic bag which may be sterilized and provided with aseptic connectors to the at least one inlet 7 and the at least one outlet 9.

1. A method for performing alkaline lysis, neutralisation and clarification of bacterial cells for extraction of plasmid DNA, said method comprising the steps of:
providing a suspension of bacterial cells and providing alkaline lysis solution into a single use mixing bag for performing an alkaline lysis of said bacterial cells, wherein said single use mixing bag comprises an integrated mixer;
mixing the content in the single use mixing bag by the integrated mixer during the alkaline lysis;
stopping the alkaline lysis by adding a neutralisation solution into the single use mixing bag;
clarifying the content in the single use mixing bag after the alkaline lysis has been performed by adding a flocculate lifting agent into the single use mixing bag, whereby at least two phases are separated inside the single use mixing bag, whereby one of said phases is a neutralised and clarified alkaline solution comprising plasmid DNA;
removing the neutralised and clarified alkaline solution from the single use mixing bag; and
filtering the neutralised and clarified alkaline solution.

2. Method according to claim 1, wherein the step of clarifying comprises adding a solution of a flocculate lifting agent comprising a salt which releases gas in acidic environment into the single use mixing bag by pumping the solution of flocculate lifting agent by a pump into an inlet of the single use mixing bag.

3. Method according to claim 2, wherein the solution of a flocculate lifting agent comprises 60-150 g AHC/L.

4. Method according to claim 1, wherein the step of providing a suspension of bacterial cells and providing alkaline lysis solution into the single use mixing bag comprises first the step of pumping a first volume of suspension of bacterial cells into the single use mixing bag and then the step of pumping a second volume of an alkaline lysis solution into the single use mixing bag, wherein said step of pumping the second volume of an alkaline lysis solution is performed during a time period between 0.5-3 minutes and wherein the step of mixing is performed during at least a part of said time period during which alkaline lysis solution is pumped into the single use mixing bag.

5. Method according to claim 1, wherein the step of stopping the alkaline lysis by adding a neutralisation solution into the single use mixing bag is performed by adding a neutralisation solution consisting of 2.5-10 M potassium acetate having a pH of 4.5-6.0 and a temperature between 1-8° C. and wherein the method further comprises the step of mixing the content in the single use mixing bag by the integrated mixer during the addition of the neutralisation solution, whereby a mixing velocity during this step of stopping the alkaline lysis is kept lower than a mixing velocity during the step of alkaline lysis in order to avoid fragmentation and/or disruption of genomic DNA.

6. Method according to claim 1, wherein a total time for the alkaline lysis counted from start of addition of alkaline lysis solution to end of addition of neutralisation solution is less than 10 minutes or less than 6 minutes.

7. Method according to claim 1, wherein the addition of alkaline lysis solution and/or the addition of neutralisation solution and/or the addition of a flocculate lifting agent into the single use mixing bag is provided via an inlet of the single use mixing bag which is positioned in a bottom part of the single use mixing bag and wherein the integrated mixer of the single use mixing bag also is provided in a bottom part of the single use mixing bag.

8. Method according to claim 1, wherein the method further comprises the step of sensing one or more parameters of the content in the single use mixing bag and/or of the content in an inlet fluid line and/or outlet fluid line connected to the single use mixing bag by one or more sensors positioned in one or more positions inside the single use mixing bag and/or in connection with the inlet fluid line and/or the outlet fluid line.

9. Method according to claim 8, wherein the method further comprises controlling pumping of contents into and out from the single use mixing bag and/or mixing velocity of the integrated mixer in the single use mixing bag based on the sensed parameters detected by the one or more sensors.

10. Method according to claim 1, further comprising the step of controlling the integrated mixer from outside the single use mixing bag by magnetism without direct contacting the mixer, whereby the integrated mixer is a magnetic impeller.

11. Method according to claim 1, further comprising the step of releasing excessive gases and potential foam produced during the step of clarifying via a vent valve device provided in the single use mixing bag.

12. Method according to claim 1, wherein said method is an automated method controlled by a control system to which said single use mixing bag and pumps are connected.

13. Method according to claim 1, wherein the single use mixing bag is a flexible, plastic bag which is sterilized and provided with aseptic connectors to at least one inlet and at least one outlet provided in the single use mixing bag.

14. Use of a single use mixing bag for performing alkaline lysis, neutralisation and clarification of bacterial cells according to claim 1, wherein said single use mixing bag comprises at least one inlet, at least one outlet, a vent valve device and an integrated mixer.

15. Use of a single use mixing bag according to claim 14, wherein said single use mixing bag is a flexible, plastic bag which is sterilized and provided with aseptic connectors to the at least one inlet and the at least one outlet.

16. A system for performing alkaline lysis, neutralisation and clarification of bacterial cells for extraction of plasmid DNA, said system comprising:
a single use mixing bag which comprises at least one inlet, at least one outlet, of which one is a clarified phase outlet, a vent valve device and an integrated mixer;
at least one inlet fluid line connected to at least one of the at least one inlet of the single use mixing bag and having at least one connector for connection to fluid sources comprising fluids to be added to the single use mixing bag
at least one inlet pump connected to the at least one inlet fluid line;
an outlet fluid line connected to the clarified phase outlet of the single use mixing bag;
at least one outlet pump connected to the outlet fluid line;
a collection bag connected to the outlet fluid line for collection of a neutralised and clarified alkaline solution removed from the single use mixing bag via the clarified phase outlet;
a filter connected to the outlet fluid line and provided between the clarified phase outlet and the collection bag such that a content removed from the clarified phase outlet of the single use mixing bag is filtered through the filter on its way to the collection bag; and
a control system connected to the at least one inlet pump, the at least one outlet pump and to a mixer controller which can control the integrated mixer of the single use mixing bag, wherein said control system is configured for controlling the at least one inlet pump, the at least one outlet pump and the integrated mixer in the single use mixing bag to pump content into the single use mixing bag, pump content out from the single use mixing bag and mix the content in the single use mixing bag according to the method of claim 1.

17. System according to claim 16, wherein said single use mixing bag is a flexible, plastic bag which is sterilized and provided with aseptic connectors to at least one inlet and at least one outlet.

18. System according to claim 16, wherein said single use mixing bag comprises at least one sensor which is configured for sensing one or more parameters of the content in the single use mixing bag, such as one or more of pH, temperature, turbidity and conductivity.

19. System according to claim 18, wherein the control system is configured for controlling the at least one outlet pump to pump content out from the single use mixing bag based on a sensed property from a sensor positioned in vicinity of the clarified phase outlet.
